(19) United States
(12) Patent Application Publication
(10) Pub. No.: US 2024/0285548 A1
(43) Pub. Date: Aug. 29, 2024

(54) AGENT FOR PREVENTION AND/OR TREATMENT OF COGNITIVE IMPAIRMENT

(71) Applicant: NATIONAL CEREBRAL AND CARDIOVASCULAR CENTER, Osaka (JP)

(72) Inventors: Yorito HATTORI, Osaka (JP); Masafumi IHARA, Osaka (JP)

(73) Assignee: NATIONAL CEREBRAL AND CARDIOVASCULAR CENTER, Osaka (JP)

(21) Appl. No.: 18/376,982

(22) Filed: Oct. 5, 2023

(30) Foreign Application Priority Data
Oct. 7, 2022 (JP) 2022-162718

(51) Int. Cl. A61K 31/05; A61P 25/28

(52) U.S. Cl. CPC A61K 31/05; A61P 25/28

(57) ABSTRACT

This invention provides a novel preventive and/or therapeutic agent for vascular cognitive impairment, comprising, as an active ingredient, a compound that has not previously been demonstrated to have a preventive and/or therapeutic effect on vascular cognitive impairment in humans. The invention provides a preventive and/or therapeutic agent for vascular cognitive impairment in humans, comprising a polyphenol compound.

### TECHNICAL FIELD

The present invention relates to a preventive and/or therapeutic agent for cognitive impairment.

### BACKGROUND ART

The number of people aged 65 and older with dementia is estimated to be about 6 million as of 2020 in Japan, and it is predicted that about 7 million people (about 1 in 5 elderly people) will have dementia by 2025. Therefore, approaches toward dementia care are considered to become increasingly important for Japan's aging society in the future.

A condition that is not so severe as to interfere with daily living as in dementia, but is neither considered normal nor dementia, with memory and other abilities being impaired, is called “mild cognitive impairment” (MCI). Research has shown that about half of those with MCI will develop dementia within 5 years. Thus, initiating preventive activities from the MCI stage is expected to slow the progression of dementia.

However, research conducted on therapeutic agents for dementia and mild cognitive impairment often shows cases in which drugs that are found to be effective in animal experiments are not found to be effective in humans, making their development difficult.

### CITATION LIST

### Non-Patent Literature

NPL 1: Hattori Y, Okamoto Y, Maki T, Yamamoto Y, Oishi N, Yamahara K, et al. Silent information regulator 2 homolog 1 counters cerebral hypoperfusion injury by deacetylating endothelial nitric oxide synthase. Stroke. 2014; 45 (11): 3403-11.
NPL 2: Hattori Y, Okamoto Y, Nagatsuka K, Takahashi R, Kalaria RN, Kinoshita M, et al. SIRT1 attenuates severe ischemic damage by preserving cerebral blood flow. Neuroreport. 2015; 26 (3): 113-7.
NPL 3: Wang N, He J, Pan C, Wang J, Ma M, Shi X, Xu Z. Resveratrol Activates Autophagy via the AKT/mTOR Signaling Pathway to Improve Cognitive Dysfunction in Rats With Chronic Cerebral Hypoperfusion. Front Neurol. 2019; 13: 859.
NPL 4: Researching Alzheimer's Medicines. Setbacks and Stepping Stones. PhRMA Analysis of Adis R and D Insight Database. 2021.
NPL 5: Turner R S, Thomas R G, Craft S, van Dyck C H, Mintzer J, Reynolds B A, Brewer J B, Rissman R A, Raman R, Aisen P S. A randomized, double-blind, placebo-controlled trial of resveratrol for Alzheimer disease. Neurology. 2015; 85 (16): 1383-91.

### SUMMARY OF INVENTION

### Technical Problem

A problem to be solved by the present invention is to provide a novel preventive and/or therapeutic agent for vascular cognitive impairment, comprising, as an active ingredient, a compound that has not previously been demonstrated to have a preventive and/or therapeutic effect on vascular cognitive impairment in humans.

### Solution to Problem

In such circumstances, the present inventors studied a wide variety of compounds and consequently found that humans administered with a polyphenol compound showed an improvement in cerebral blood flow and showed a trend toward improvement in cerebral oxygen metabolic rate, and further that humans administered with a polyphenol compound showed an improvement in cognitive functions, such as delayed recall, memory domains (delayed recall, orientation, teaching recovery), and executive functions/spatial cognitive functions, and also showed a trend toward improvement in recognition. The present invention is based on such novel findings.

Accordingly, the present invention provides the following items.

Item 1. A preventive and/or therapeutic agent for vascular cognitive impairment in humans, comprising a polyphenol compound.

Item 2. The preventive and/or therapeutic agent for vascular cognitive impairment according to Item 1, wherein the polyphenol compound is administered in an amount of 1 to 300 mg per day.

Item 3. The preventive and/or therapeutic agent for vascular cognitive impairment according to Item 1, wherein the polyphenol compound is administered in an amount of 10 to 50 mg per day.

Item 4. The preventive and/or therapeutic agent for vascular cognitive impairment according to any one of Items 1 to 3, wherein the polyphenol compound comprises a resveratrol compound or a salt thereof.

Item 5. The preventive and/or therapeutic agent for vascular cognitive impairment according to any one of Items 1 to 4, wherein the polyphenol compound comprises resveratrol or a salt thereof.

Item 6. The preventive and/or therapeutic agent for vascular cognitive impairment according to any one of Items 1 to 5, which is an agent for oral administration.

Item 7. A preventive and/or therapeutic agent for vascular cognitive impairment caused by carotid artery stenosis/occlusion, comprising a polyphenol compound.

Item 8. A preventive and/or therapeutic agent for vascular cognitive impairment in humans, comprising a polyphenol compound that activates SIRT1.

Item 9. A preventive and/or therapeutic agent for vascular cognitive impairment in humans caused by carotid artery stenosis/occlusion, comprising a polyphenol compound that activates SIRT1.

### Advantageous Effects of Invention

The present invention can provide a novel preventive and/or therapeutic agent for vascular cognitive impairment, comprising, as an active ingredient, a compound that has not previously been demonstrated to have a preventive and/or therapeutic effect on vascular cognitive impairment in humans.

The present inventors reported the findings that the NAD-dependent longevity gene SIRT1 contributes to an improvement in cognitive function through the following mechanism in basic research by using carotid artery stenosis model mice (Non-patent Literature (NPL) 1), and that SIRT1 suppresses delayed neuronal death in the hippocampus in carotid artery occlusion model mice (NPL 2). There has also been a report that the administration of resveratrol to carotid artery occlusion model mice caused activation of autophagy and reduced oxidative stress-induced nerve damage, showing an improvement in cognitive function (NPL 3). However, as stated above, there are many cases in which drugs that are found to be effective in animal experiments are not found to be effective in humans (NPL 4); thus, it is common technical knowledge in the field of prevention and treatment of cognitive function impairment that the results of animal experiments cannot always be extrapolated to humans.

Furthermore, it has also been reported that no effect was observed even when 500 mg of resveratrol was administered to human Alzheimer's patients (NPL 5). However, the present inventors found that resveratrol shows a therapeutic effect in treating human patients with vascular cognitive impairment. Accordingly, such an effect of the present invention cannot be expected from previously known techniques.

### DESCRIPTION OF EMBODIMENTS

Preventive and/or Therapeutic Agent for Vascular Cognitive Impairment

The present invention provides a preventive and/or therapeutic agent for vascular cognitive impairment in humans, comprising a polyphenol compound. In the present specification, examples of polyphenol compounds include tannic acid, isoflavones, catechin, curcumin, tannins, hydroxybenzoic acid, hydroxycinnamic acid, flavonoids, lignans, stilbenes, caffeic acid, chlorogenic acid, anthocyanins, pyrogallol, ellagic acid, gallic acid, theaflavin-3-gallate, resveratrol, kaempferol, quercetin, myricetin, luteolin, delphinidin, cyanidin, ampelopsin, hesperidin, aurantinidin, europinidine, pelargonidin, malvidin, peonidin, petunidin, and rosinidin, and salts thereof. These polyphenol compounds can be used alone or in a combination of two or more. The polyphenol compound is preferably resveratrol, curcumin, gallic acid, a derivative thereof, a salt thereof, or the like. Resveratrol and derivatives thereof may be collectively referred to simply as a “resveratrol compound.” Examples of derivatives of resveratrol include compounds (e.g., piceatannol) in which one or more (e.g., 1 to 3, or 1) hydroxyl groups and/or alkyl groups (e.g., methyl, ethyl, and propyl) etc. are substituted with respect to resveratrol. Thus, in a preferred embodiment, the present invention provides a preventive and/or therapeutic agent for vascular cognitive impairment in humans, comprising a resveratrol compound or a salt thereof.

In the present invention, vascular cognitive impairment refers to a concept that encompasses mild cognitive function impairment associated with cerebral vascular disease, as well as vascular dementia. Thus, the preventive and/or therapeutic agent of the present invention can be used for the prevention and/or treatment of, for example, vascular dementia and vascular mild cognitive impairment, and typically vascular cognitive impairment at a mild cognitive impairment level. Furthermore, the preventive and/or therapeutic agent of the present invention can also be used to suppress the deterioration of vascular cognitive impairment (typically vascular cognitive impairment at a mild cognitive impairment level).

In the present invention, vascular cognitive impairment includes vascular cognitive impairment caused by carotid artery stenosis/occlusion. Carotid artery stenosis/occlusion may be symptomatic or asymptomatic. Carotid artery stenosis/occlusion is a major causative disease of cerebral infarction; carotid artery stenosis/occlusion causes cognitive function impairment even without developing cerebral infarction, and is one of the causative diseases of vascular cognitive impairment. In terms of the risk of developing cerebral infarction, ACAS, which is large-scale clinical experiments conducted in the United States and Canada on patients with asymptomatic carotid artery stenosis, has reported that the 5-year risk of cerebral infarction during medical treatment (aspirin and risk factor management) is 11.0% in patients with moderate or severe carotid artery stenosis (stenotic diameter: 60% or more). Further, in terms of the risk of developing cognitive function impairment, changes in cognitive function were evaluated in patients with asymptomatic severe carotid artery stenosis (210 cases) for a prospective 36-month period, which resulted in many patients having deteriorated cognitive function (3 scores or more decreased in the mini-mental state examination) compared with healthy adults; patients with asymptomatic moderate or severe carotid artery stenosis (stenotic diameter: 50% or more) (82 cases) showed deterioration in cognitive function compared with the control group; furthermore, patients with moderate or severe carotid artery stenosis (25 cases) had significantly deteriorated Stroop test results compared with the control group. Asymptomatic carotid artery stenosis/occlusion is one of the representative causative diseases of vascular cognitive impairment.

At present, no effective treatment methods to improve cognitive function for carotid artery stenosis/occlusion have been established, and the development of a novel treatment method is hoped for. The present invention can be used as a preventive and/or therapeutic agent for carotid artery stenosis/occlusion.

In a preferred embodiment, the polyphenol compound for use in the present invention activates SIRT1. In carotid artery stenosis/occlusion, which is a cause of vascular cognitive impairment, vascular endothelial injury plays an important role; the decrease in the expression of vascular endothelial nitric oxide synthase (eNOS) causes a decrease in vascular endothelial function, which causes the development of carotid artery stenosis/occlusion. eNOS not only improves blood flow through NO-induced vasodilation, but is also involved in the repair of vascular endothelial injury and further activates eNOS itself. Accordingly, the present inventors focused on a polyphenol compound that has an action of activating the NAD⁺/SIRT1 pathway as a novel treatment method for increasing the cerebral blood flow by targeting the vascular endothelium. For example, trans-resveratrol shows pharmacological action by binding to phosphodiesterase 4 to inhibit the activity, causing CAMP increase. The CAMP increase activates the CamKKβ-AMPK pathway, resulting in increased intracellular NAD⁺ and increased SIRT1 activity. SIRT1 then converts eNOS into its active form via deacetylation, causing cerebral vasodilation with the eNOS-NO axis.

In a preferred embodiment, the present invention includes resveratrol [CAS No. 501-36-0 (3,4′, 5-trihydroxy-trans-stilbene)], and more preferably a known substance having the following structure.

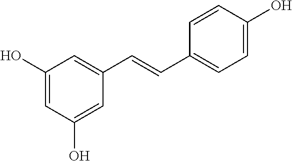 [SMILES: Oc1ccc(/C=C/c2cc(O)cc(O)c2)cc1]

Examples of derivatives of resveratrol include, but are not limited to, trans and cis isomers, trans- and cis-isomer mixtures, dimers and other polymers, glycosides, and methylated resveratrol. Specific and non-limiting examples of resveratrol derivatives include resveratrol-4′-O-β-D-glucopyranoside, 3,5,4′-trans-trihydroxystilbene, 3,5,3′-trans-trihydroxystilbene, 3,5-trans-dihydroxystilbene, 3,4′-trans-dihydroxystilbene, 3,3′ . . . trans-dihydroxystilbene, 3-trans-hydroxystilbene, 4-trans-hydroxystilbene, 3,4,3′, 5′-trans-tetrahydroxystilbene, and 3,5-dimethoxy-4′-hydroxy-trans-stilbene.

In the present invention, the resveratrol compound may be used in a free form or in the form of a pharmaceutically acceptable salt. When a salt of the resveratrol compound is used as the active ingredient of the present invention, the salt of the resveratrol compound can include acid addition salts and salts with bases. Specific examples of the acid addition salts include inorganic acid salts, such as hydrochloride, hydrobromide, hydroiodide, sulfate, perchlorate, and phosphate; organic acid salts, such as oxalate, malonate, succinate, maleate, fumarate, lactate, malate, citrate, tartrate, benzoate, trifluoroacetate, acetate, methanesulfonate, p-toluenesulfonate, and trifluoromethanesulfonate; and acidic amino acid salts, such as glutamate and aspartate. Specific examples of salts with bases include alkali metal or alkaline earth metal salts, such as sodium, potassium, and calcium salts; salts with organic bases, such as pyridine and triethylamine salts; and salts with basic amino acids, such as lysine and arginine.

In the present invention, resveratrol is preferred for use from among the resveratrol compound or a salt thereof.

The polyphenol compound, which is the active ingredient of the present invention, may be sometimes present in the form of a hydrate or a solvate; thus, such a hydrate and solvate are also encompassed by the compounds that serve as the active ingredient of the present invention.

Examples of solvents that form a solvate include alcohols, such as ethanol and propanol; organic acids, such as acetic acid; esters, such as ethyl acetate; ethers, such as tetrahydrofuran and diethyl ether; ketones, such as acetone; and DMSO. These solvents can be used alone or as a solvent mixture of two or more solvents.

In the present invention, the preventive and/or therapeutic agent for vascular cognitive impairment in humans, comprising a polyphenol compound includes pharmaceuticals, food products (e.g., foods with health claims (foods with nutritional claims, foods for specified health uses, foods with functional claims)), and the like. Examples of the form of a food product include supplements.

In the present invention, the polyphenol compound, which is the active ingredient of the present invention, may be used as is as a preventive and/or therapeutic agent for vascular cognitive impairment in humans, or may be used as a composition obtained by combining with various carriers that are pharmaceutically acceptable or that can be added to food products (e.g., tonicity agents, chelating agents, stabilizers, pH adjusters, preservatives, antioxidants, solubilizing agents, and thickening agents).

Examples of tonicity agents include sugars, such as glucose, trehalose, lactose, fructose, mannitol, xylitol, and sorbitol; polyhydric alcohols, such as glycerin, polyethylene glycol, and propylene glycol; and inorganic salts, such as sodium chloride, potassium chloride, and calcium chloride. These tonicity agents can be used alone or in a combination of two or more.

Examples of chelating agents include edetate salts, such as disodium edetate, disodium calcium edetate, trisodium edetate, tetrasodium edetate, and calcium edetate, ethylenediaminetetraacetate, nitrilotriacetic acid or a salt thereof, sodium hexametaphosphate, and citric acid. These chelating agents can be used alone or in a combination of two or more.

Examples of stabilizers include sodium hydrogen sulfite.

Examples of pH adjusters include acids, such as hydrochloric acid, carbonic acid, acetic acid, and citric acid; alkali metal hydroxides, such as sodium hydroxide and potassium hydroxide; alkali metal carbonates or hydrogen carbonates, such as sodium carbonate; alkali metal acetates, such as sodium acetate; alkali metal citrates, such as sodium citrate; and bases, such as trometamol. These pH adjusters can be used alone or in a combination of two or more.

Examples of preservatives include sorbic acid, potassium sorbate, methyl paraoxybenzoate, ethyl paraoxybenzoate, propyl paraoxybenzoate, butyl paraoxybenzoate, and other paraoxybenzoic acid esters, chlorhexidine gluconate, benzalkonium chloride, benzethonium chloride, cetylpyridinium chloride, and other quaternary ammonium salts, alkyl polyaminoethylglycine, chlorobutanol, polyquad, polyhexamethylene biguanide, and chlorhexidine. These preservatives can be used alone or in a combination of two or more.

Examples of antioxidants include sodium hydrogen sulfite, dry sodium sulfite, sodium pyrosulfite, and concentrated mixed tocopherols. These antioxidants can be used alone or in a combination of two or more.

Examples of solubilizing agents include sodium benzoate, glycerin, D-sorbitol, glucose, propylene glycol, hydroxypropylmethylcellulose, polyvinylpyrrolidone, macrogol, and D-mannitol. These solubilizing agents can be used alone or in a combination of two or more.

Examples of thickening agents include polyethylene glycol, methylcellulose, ethylcellulose, carmellose sodium, xanthan gum, sodium chondroitin sulfate, hydroxyethylcellulose, hydroxypropylcellulose, hydroxypropylmethylcellulose, polyvinylpyrrolidone, and polyvinyl alcohol. These thickening agents can be used alone or in a combination of two or more.

In addition to the polyphenol compound, the composition above may further comprise a compound that is considered to have a preventive or therapeutic action on vascular cognitive impairment, or a compound known to have a preventive or therapeutic action on at least one of the causes of vascular cognitive impairment. Examples of such compounds include aspirin, cilostazol, clopidogrel, prasugrel, warfarin, a selective direct-acting factor Xa inhibitor, and a direct thrombin inhibitor, which have a preventive or therapeutic action on ischemic cerebral vascular disease, which is one of the causes of vascular cognitive impairment; and an anti-acetylcholinesterase inhibitor and NMDA receptor antagonist, which are currently approved anti-dementia drugs. These compounds can be used alone or in a combination of two or more.

In an embodiment of the composition, the content of the polyphenol compound in the composition is not limited, and can be suitably set, for example, from conditions such as 90 mass % or more, 70 mass % or more, 50 mass % or more, 30 mass % or more, 10 mass % or more, 5 mass % or more, and 1 masse or more.

Examples of formulation forms include, but are not limited to, various formulation forms, including agents for oral administration, such as tablets, pills, capsules, powders, granules, and syrups; and agents for parenteral administration, such as injections (e.g., intravenous injection, intramuscular injection, and local injection), gargles, drips, agents for external use (ointments, creams, skin patches, inhalers), and suppositories. Among these, preferred examples of formulation forms include agents for oral administration (e.g., tablets, pills, capsules, powders, granules, and syrups).

In the present invention, the administration amount of the polyphenol compound varies depending on the administration route, patient's age, body weight, symptoms, etc., and cannot be unconditionally defined. The daily administration amount for adults may be usually set to about 1000 mg or less, preferably about 300 mg or less, more preferably 50 mg or less, and even more preferably 35 mg or less. The lower limit of the administration amount of the polyphenol compound is also not limited, and, for example, the daily administration amount for adults can be suitably set within the ranges of usually 1 mg or more, preferably 10 mg or more, and more preferably 25 mg or more. Typically, the daily administration amount for adults is 30 mg. When the administration is performed once a day, the administration amount above may be contained in a single formulation, and when the administration is performed three times a day, one-third of the administration amount above may be contained in a single formulation. Further, in the present invention, when a resveratrol compound or a salt thereof is used as the polyphenol compound, the administration amount of the resveratrol compound or a salt thereof varies depending on the administration route, patient's age, body weight, symptoms, etc; and cannot be unconditionally defined. The daily administration amount of the resveratrol compound for adults may be usually set to about 1000 mg or less, preferably about 300 mg or less, more preferably 50 mg or less, and even more preferably 35 mg or less. The lower limit of the administration amount of the resveratrol compound or a salt thereof is also not limited, and, for example, the daily administration amount of the resveratrol compound for adults can be suitably set within the ranges of usually 1 mg or more, preferably 10 mg or more, and more preferably 25 mg or more. Typically, the daily administration amount for adults is 30 mg. When the administration is performed once a day, the administration amount above may be contained in a single formulation, and when the administration is performed three times a day, one-third of the administration amount above may be contained in a single formulation.

The preventive and/or therapeutic agent for vascular cognitive impairment in humans of the present invention prevents and/or treats vascular cognitive impairment in humans at least by increasing cerebral blood flow. Thus, the present invention also provides a cerebral blood flow promoter for humans, comprising the polyphenol compound. Further, as demonstrated in the Examples below, a polyphenol compound improves cognitive functions in humans, such as delayed recall, recognition, memory domains (delayed recall, orientation, recognition, teaching recovery), and executive functions/spatial cognitive functions, in particular, delayed recall, memory domains (delayed recall, orientation, recognition, teaching recovery), and executive functions/spatial cognitive functions. Accordingly, in a preferred embodiment, the present invention provides a polyphenol compound-containing agent for improving at least one cognitive function in humans selected from the group consisting of delayed recall, recognition, memory domains, executive function, and spatial cognitive function, and preferably at least one cognitive function selected from the group consisting of executive function and spatial cognitive function. In the present invention, the expression “improvement in cognitive function” etc. encompasses not only increasing cognitive function, but also preserving cognitive function (i.e., suppressing the deterioration of cognitive function).

The active ingredient, formulation form, administration amount, etc. of the promoter or agent above are the same as those of the preventive and/or therapeutic agent for vascular cognitive impairment in humans.

The present invention is described in more detail below with reference to Examples; however, the present invention is not limited thereto.

### EXAMPLES

### Example 1

### 1-I. Method

Subjects were patients with asymptomatic moderate or severe carotid artery stenosis/occlusion who visited the National Cerebral and Cardiovascular Center in Japan from July 2020 to August 2021 and who were taking or not taking trans-resveratrol in an amount of 30 mg/day. The inclusion criteria were as follows: (1) patients with asymptomatic moderate or severe carotid artery stenosis/occlusion who presented with a peak systolic velocity of 130 cm/s or more at a stenotic lesion on carotid ultrasonography (corresponding to a stenotic diameter of >50%) and who underwent test (2) and/or (3); (2) patients who underwent cognitive function tests (Alzheimer's Disease Assessment Scale-Cognitive subscale 13 (ADAS-Cog) and Montreal Cognitive Assessment (MoCA)) longitudinally twice; (3) patients who also underwent ¹⁵O-gas positron emission tomography (PET) longitudinally twice in a similar manner. The primary outcome is a comparison of the amounts of change in ADAS-Cog during the observation period (differential analysis). The secondary outcomes are (1) a comparison of the amounts of change in MoCA during the observation period (differential analysis), and (2) a comparison of the amounts of change in cerebral blood flow on ¹⁵O -gas PET (differential analysis). This study was initiated after receiving approval from the Research Ethics Review Committee of the National Cerebral and Cardiovascular Center (Research Project Number: R20113).

### ¹⁵O-gas PET Quantitative Determination

All PET images were anatomically standardized using linear and nonlinear transformations with SPM2 to ensure that the brain images of all patients had the same anatomical format, and the cerebral blood flow was quantified using 3DSRT (PDRadiopharma Inc., Tokyo, Japan). Each hemisphere was divided into twelve ROI segments, grouped according to the arterial supply: callosomarginal artery area, pericallosal artery area, precentral artery area, central artery area, angular artery area, parietal lobe, temporal lobe, occipital lobe, hippocampus, lenticular nucleus, thalamus, and cerebellum. The frontal circulation areas were defined to be the callosomarginal artery area, pericallosal artery area, precentral artery area, and central artery area. In quantifying the relative cerebral blood flow, counts under each ROI were normalized to an average cerebral blood flow count within the bilateral cerebellum.

### 1-II. Results

### 1-II-1. Patient Background

Table 1 below shows the results. As shown in Table 1, 45 patients who did not take resveratrol (non-RES group) and 37 patients who took resveratrol (RES group) satisfied the inclusion criteria. The mean ages were 76.4±7.2 years in the non-RES group and 76.9±8.5 years in the RES group. The proportions of male patients were 80.0% in the non-RES group and 73.0% in the RES group. The baseline ADAS-Cog scores were 15.0±5.6 in the non-RES group and 15.7±7.2 in the RES group. The MoCA scores were 23.6±3.5 in the non-RES group and 23.7±3.6 in the RES group. For both ADAS-Cog and MoCA, no significant differences were found between the two groups. The baseline scores were under the category of mild cognitive impairment (MCI). These 82 patients were tested for cognitive function and/or cerebral blood flow with ¹⁵O-gas PET.

TABLE 1
Patient Background

| | Non-RES | | RES | |
|---|---|---|---|---|
| Number of patients | 45 | | 37 | |
| Age (years) | 76.4 ± 7.2 | | 76.9 ± 8.5 | |
| Gender (proportion of males (%) | 36 | (80.0) | 27 | (73.0) |
| Smoking (%) | 19 | (42.2) | 8 | (21.6) |
| Alcohol intake (%) | 24 | (53.3) | 16 | (43.2) |
| PMH of coronary artery disease (%) | 13 | (28.9) | 6 | (16.2) |
| Atrial fibrillation (%) | 9 | (20.0) | 3 | (8.1) |
| Antiplatelet use (%) | 28 | (62.2) | 28 | (75.7) |
| Anticoagulant use (%) | 9 | (20.0) | 2 | (5.4) |
| Antihypertensive drug use (%) | 40 | (88.9) | 29 | (78.4) |
| Statin use (%) | 41 | (91.1) | 31 | (83.8 |
| Side of carotid stenosis | | | | |
| Right | 14 | (31.1) | 10 | (27.0) |
| Left | 17 | (37.8) | 21 | (56.8) |
| Bilateral | 14 | (31.1) | 6 | (16.2) |

| | | |
|---|---|---|
| Total MoCA score | 23.6 ± 3.5 | 23.7 ± 3.6 |
| Total ADAS-Cog score | 15.0 ± 5.6 | 15.7 ± 7.2 |

### 1-II-2. Change in Cognitive Function

Table 2 below shows the results. As shown in Table 2, the results of MoCA showed a significant association between RES intake and improvement in cognitive function in executive functions/spatial cognitive functions. In addition, the results of changes in cognitive function as measured by ADAS-Cog showed a significant association between RES intake and improvement in cognitive functions in terms of delayed recall, memory domains (delayed recall, orientation, recognition, and teaching recovery), and total scores (Table 2).

TABLE 2
Amount of change in MoCA and ADAS-Cog

| | Non-RES | RES | p value |
|---|---|---|---|
| MoCA | | | |
| Executive function/spatial | −0.22 ± 1.2 | 0.35 ± 1.0 | 0.022 |
| cognitive function | | | |
| ADAS-Cog | | | |
| Memory domain | 0.40 ± 3.3 | −1.65 ± 3.0 | 0.005 |
| Delayed recall | 0.40 ± 1.7 | −0.89 ± 1.7 | 0.001 |
| Total | 0.46 ± 3.8 | −1.48 ± 3.4 | 0.018 |

### 1-II-3. Change in Cerebral Blood Flow

Table 3 below shows the results. As shown in Table 3, the cerebral blood flow, cerebral oxygen metabolic rate, and cerebral oxygen uptake rate were measured with ¹⁵O-gas PET. Using 3D-SRT (FUJIFILM RI Pharma, Tokyo, Japan), the difference in the amount of change was analyzed (for the RES group, the difference between the cerebral blood flow before RES administration (relative to cerebellum) and the cerebral blood flow after RES administration (relative to cerebellum); for the non-RES group, the cerebral blood flow at intervals of 6 months to 1 year (relative to cerebellum)). The difference in the cerebral blood flow before and after RES administration showed a significant association between RES intake and an increase in the cerebral blood flow in the bilateral callosomarginal artery areas, right frontal lobe, left putamen, and bilateral thalamus. In other areas as well, there was a tendency for an association between RES intake and an increase in the cerebral blood flow (Table 3). For cerebral oxygen metabolic rate, there was a tendency for an association between RES intake and an increase in the cerebral oxygen metabolic rate in all areas. In both of the groups, the cerebral oxygen uptake rate showed no association.

TABLE 3
Amount of change in cerebral blood flow (relative to cerebellum)

| | Non-RES | RES | p value |
|---|---|---|---|
| Right callosomarginal artery | −0.004 ± 0.41 | 0.179 ± 0.34 | 0.013 |
| area | | | |
| Left callosomarginal artery area | −0.003 ± 0.40 | 0.171 ± 0.42 | 0.031 |
| Right frontal lobe | 0.002 ± 0.40 | 0.215 ± 0.35 | 0.028 |
| Left putamen | 0.000 ± 0.47 | 0.19 ± 0.57 | 0.009 |
| Right thalamus | −0.001 ± 0.85 | 0.393 ± 0.85 | 0.035 |
| Left thalamus | −0.108 ± 0.95 | 0.449 ± 0.90 | 0.010 |

### Example 2

In Example 1 above, the 82 subject patients included patients who were tested for both change in cognitive function and change in cerebral blood flow, as well as patients who were tested for only either change in cognitive function or change in cerebral blood flow. Therefore, based on the above results, the same analysis as in Example 1 was performed again on 79 patients who were tested for both change in cognitive function and change in cerebral blood flow from among the 82 patients.

### 2-1. Patient Background

Among the 79 patients, 36 patients took resveratrol at 30 mg/day and received the best medical treatment (RES group), while 43 patients received the best medical treatment as well but did not take resveratrol (non-RES group). The results for the 79 patients are shown in Table 4 below. The mean age (76.4±7.4 vs. 77.0±8.6 years) and the proportion of male patients [34 (79.1%) vs. 27 (75.0%)] were comparable between the resveratrol and non-resveratrol groups. The baseline mean total MoCA scores were 23.6±3.6 in the non-RES group and 23.7±3.6 in the RES group. Further, the mean total ADAS-Cog scores were 14.6±5.4 in the non-RES group and 15.8±7.2 in the RES group. The mean observation periods were 244.8±86.9 and 221.2±61.9 days, respectively (p=0.18). The baseline scores were under the category of mild cognitive impairment (MCI) (Table 4).

TABLE 4
Patient Background

| | Non-RES group | | RES group | |
|---|---|---|---|---|
| | (n = 43) | | (n = 36) | |
| Age | 76.4 | (±7.4) | 77.0 | (±8.6) |
| Male (%) | 34 | (79.1) | 27 | (75.0) |
| Smoking (%) | 18 | (41.9) | 8 | (22.2) |
| Alcohol intake (%) | 22 | (51.2) | 16 | (44.4) |
| PMH of coronary artery disease (%) | 12 | (27.9) | 6 | (16.7) |
| Atrial fibrillation (%) | 9 | (20.9) | 3 | (8.3) |
| Antiplatelet use (%) | 26 | (60.5) | 27 | (75.0) |
| Anticoagulant use (%) | 9 | (20.9) | 2 | (5.6) |
| Antihypertensive drug use (%) | 39 | (90.7) | 29 | (80.6) |
| Statin use (%) | 39 | (90.7) | 30 | (83.3) |
| Side of carotid stenosis | | | | |
| Right | 13 | (30.2) | 10 | (27.8) |
| Left | 17 | (39.5) | 20 | (55.6) |
| Bilateral | 13 | (30.2) | 6 | (16.7) |
| Total MoCA score | 23.6 | (±3.6) | 23.7 | (±3.6) |
| Total ADAS-Cog score | 14.6 | (±5.4) | 15.8 | (±7.2) |
| Observation period (days) | 244.8 | (±86.9) | 221.2 | (±61.9) |

### 2-2. Change in Cognitive Function

For the 79 patients, the amount of change in MoCA and ADAS-Cog scores was examined to investigate changes in cognitive function associated with administration of resveratrol. Resveratrol intake was found to be significantly associated with improvement in visuospatial cognitive and executive functions based on MoCA (non-RES group: −0.2±1.3 vs. RES group: 0.4±0.9; p=0.020). However, the total MoCA scores did not show a significant difference (−0.4±3.2 vs. 0.4±2.6). According to ADAS-Cog, a significant association was found with cognitive function improvement in the RES group in terms of delayed recall (0.5±1.6 VS. −0.9±1.8; p=0.001), memory domain (0.4±3.3 VS. −1.6±3.1; p=0.007), and total score (0.5±3.7 vs. −1.5±3.4; p=0.019). Therefore, after adjusting for age, gender, hypertension, diabetes, dyslipidemia, and coronary artery disease, a multivariate linear regression model analysis was performed. Resveratrol intake was an independent predictor of improvement in spatial cognitive and executive functions (β=0.26, 95% confidence interval [CI]: 0.062-1.15, p=0.03) and memory (memory domain: β=−0.26, 95% CI: −3.23 to −0.15, p=0.032; delayed recall: β=−0.37, 95% CI: −2.16 to −0.54, p=0.001) (Table 5). Accordingly, resveratrol may have contributed to improvement in the visuospatial cognitive function, executive function, and memory.

TABLE 5
Association between resveratrol intake and cognitive
function change (linear regression analysis)

| | Univariate analysis | | | Multivariate analysis | | |
|---|---|---|---|---|---|---|
| | β | 95% CI | p | β | 95% CI | p |
| MoCA | | | | | | |
| Visuospatial cognitive | 0.257 | 0.086-1.10 | 0.022 | 0.26 | 0.062-1.15 | 0.030 |
| function/executive function | | | | | | |
| ADAS-Cog | | | | | | |
| Memory domain | −0.30 | −3.41 to −0.55 | 0.007 | −0.26 | −3.23 to −0.15 | 0.032 |
| Delayed recall | −0.37 | −2.12 to −0.59 | 0.001 | −0.37 | −2.16 to −0.54 | 0.001 |
| Total | −0.26 | −3.57 to −0.32 | 0.019 | −0.18 | −2.94 to −0.32 | 0.11 |

### 2-3. Change in Cerebral Blood Flow

To identify the reason associated with significant improvements in the visuospatial cognitive function, executive function, and memory in the RES group, cerebral blood flow was measured. Initially, the cerebral blood flow change in each entire hemisphere was analyzed. Compared with the non-RES group, the RES group showed a trend toward CBF improvement in the entire right hemisphere (0.0011±0.034 vs. 0.016±0.032; p=0.051) and the entire left hemisphere (−0.0011±0.035 vs. 0.014±0.031; p=0.054). To examine the changes in detail, the cerebral blood flow was analyzed in terms of the frontal lobe, basal ganglia, and thalamus, which govern the visuospatial cognitive and executive functions, and memory. Longitudinally, long-term resveratrol intake was associated with a significant increase in cerebral blood flow in the right frontal lobe (non-RES group: 0.001±0.037, RES group: 0.019±0.033; p=0.027)). In the frontal lobe, the right (−0.004±0.041 vs. 0.018±0.034; p=0.013) and left (−0.003±0.040 vs. 0.017±0.042; p=0.031) callosomarginal artery areas, and the right precentral artery area (0.002±0.040 vs. 0.021±0.035; p=0.028) showed associations between significant increase in cerebral blood flow and long-term resveratrol intake. Furthermore, the left lenticular nucleus (−0.002±0.06 vs. 0.033±0.06; p=0.01), right thalamus (−0.002±0.085 vs. 0.039±0.85; p=0.04), and left thalamus (−0.011±0.095 vs. 0.045±0.090; p=0.01) also showed significant associations. To assess the robustness of the results, multivariate linear regression models were used after adjusting for age, gender, hypertension, diabetes, dyslipidemia, and coronary artery disease, which revealed that long-term resveratrol intake resulted in the following: right frontal lobe (β=0.25, 95% CI: 0.001-0.035, p=0.039), right callosomarginal artery area (β=0.29, 95% CI: 0.005-0.041, p=0.013), right precentral artery area (β=0.25, 95% CI: 0.001-0.037, p=0.042), left lenticular nucleus (β=0.31, 95% CI: 0.009-0.067, p=0.011), and left thalamus (β=0.29, 95% CI: 0.011-0.10, p=0.016) (Table 6). Therefore, long-term resveratrol intake may contribute not only to improvement of cognitive function but also to increase in cerebral blood flow. There were no adverse events observed.

TABLE 6
Association between resveratrol intake and cerebral
blood flow change (linear regression analysis)

| | Univariable analysis | | | Multivariable analysis | | |
|---|---|---|---|---|---|---|
| Regions of interest | β | 95% CI | p | β | 95% CI | p |
| Frontal circulation area | 0.25 | 0.002-0.034 | 0.027 | 0.25 | 0.001-0.035 | 0.039 |
| Right callosomarginal artery area | 0.28 | 0.005-0.039 | 0.013 | 0.29 | 0.005-0.041 | 0.013 |
| Left callosomarginal artery area | 0.24 | 0.002-0.039 | 0.031 | 0.27 | 0.003-0.042 | 0.027 |
| Right precentral artery | 0.25 | 0.002-0.036 | 0.028 | 0.25 | 0.001-0.037 | 0.042 |
| Left lenticular nucleus | 0.29 | 0.009-0.062 | 0.009 | 0.31 | 0.009-0.067 | 0.011 |
| Thalamus | | | | | | |
| Right | 0.24 | 0.003-0.079 | 0.035 | 0.23 | 0.00-0.082 | 0.052 |
| Left | 0.29 | 0.014-0.097 | 0.010 | 0.29 | 0.011-0.10 | 0.016 |

### DISCUSSION

This study showed associations between (1) resveratrol and increased cerebral blood flow, and (2) resveratrol and improvement in cognitive function (in particular, memory, executive function, and spatial cognitive function). Resveratrol is believed to inhibit CAMP degradation by inhibiting phosphodiesterases, activate the NAD⁺/SIRT1 pathway, convert eNOS into the active form to improve cerebral blood flow through cerebral vasodilation, and finally contribute to cognitive function improvement.

### CONCLUSION

The polyphenol compound comprising resveratrol is significantly associated with the prevention of cognitive function deterioration due to vascular cognitive impairment caused by carotid artery stenosis/occlusion, and the preventive effect for vascular cognitive impairment is sufficiently promising.

1. A method for preventing and/or treating vascular cognitive impairment in humans, comprising administering an effective amount of a polyphenol compound to a human subject in need thereof.

2. The method according to claim 1, wherein the polyphenol compound is administered in an amount of 1 to 300 mg per day.

3. The method according to claim 1, wherein the polyphenol compound is administered in an amount of 10 to 50 mg per day.

4. The method according to claim 1, wherein the polyphenol compound comprises a resveratrol compound or a salt thereof.

5. The method according to claim 1, wherein the polyphenol compound comprises resveratrol or a salt thereof.

6. The method according to claim 1, wherein the polyphenol compound is administered orally.
